(19) United States
(12) Patent Application Publication
(10) Pub. No.: US 2009/0005851 A1
(43) Pub. Date: Jan. 1, 2009

(54) ENDOSCOPIC ANEURYSM REPAIR

(76) Inventor: Vicken N. Pamoukian, New York, NY (US)

Correspondence Address: THE FARRELL LAW FIRM, P.C., 333 EARLE OVINGTON BOULEVARD, SUITE 701, UNIONDALE, NY, 11553, US

(21) Appl. No.: 12/131,799

(22) Filed: Jun. 2, 2008

Related U.S. Application Data
(60) Provisional application No. 60/932,609, filed on Jun. 1, 2007.

(51) Int. Cl. A61F 2/84

(52) U.S. Cl. 623/1.11; 128/898; 606/108

(57) ABSTRACT

Provided are a method and apparatus for installing an endograft stent in an aorta, including placing the endograft stent in an aneurysm sac of the aorta with a long limb of the endograft stent in a first iliac artery of the aorta; inserting an angled end of an ipsilateral sheath into the long limb, through the first iliac artery, wherein the ipsilateral sheath houses a first wire, and wherein a first magnet is secured to an end of the first wire and positioned at an opening of the angled end of the ipsilateral sheath; pushing the first magnet out of the opening of the angled end of the ipsilateral sheath, by pushing the first wire through the ipsilateral sheath from an other end of the ipsilateral sheath, such that the first magnet exits the endograft stent through a gate; inserting a short sheath into aneurysm sac through a second iliac artery, wherein the short sheath houses a second wire, and wherein a second magnet is secured to an end of the second wire and positioned at an opening of the short sheath inside the aneurysm sac; and coupling the first magnet to the second magnet such that the gate is aligned with the second iliac artery.

### PRIORITY

This application claims priority to U.S. Provisional Application No. 60/932,609, filed Jun. 1, 2007, the contents of which is incorporated herein by reference.

### BACKGROUND OF THE INVENTION

1. Field of the Invention

The present invention relates to a method and apparatus for installing an endograft stent.

2. Description of the Related Art

FIG. 1 is a view of a conventional endograft stent positioned inside an artery.

One type of conventional endograft stent 50 is shaped like a pair of pants with only one leg, which corresponds to long limb 60 of FIG. 1. The endograft stent 50 is placed through a femoral artery and is aligned with a first iliac artery 40. Femoral catheters and wires (not shown) are then passed into the endograft stent 50, in order to add a second limb to the endograft stent 50 at a gate 70, through a second iliac artery 42, in order to complete the pants shape of the endograft stent 50.

The gate 70, which is an opening in the endograft stent 50 to which the second limb is connected can be have a size of 1 cm. The gate 70 lies within a 4-8 cm sac 30 of an aorta 10. The gate 70 can be hard to locate and pass a wire through, especially when working in a two-dimensional field of vision.

To date there remains a need for an apparatus and method for aligning a gate of an endograft stent with a second iliac artery, in order to complete installation of the endograft stent. It is the object of invention to provide such an apparatus and method.

### SUMMARY OF THE INVENTION

A method for installing an endograft stent in an aorta according to the present invention is provided, including placing the endograft stent in an aneurysm sac of the aorta with a long limb of the endograft stent in a first iliac artery of the aorta; inserting an angled end of an ipsilateral sheath into the long limb, through the first iliac artery, wherein the ipsilateral sheath houses a first wire, and wherein a first magnet is secured to an end of the first wire and positioned at an opening of the angled end of the ipsilateral sheath; pushing the first magnet out of the opening of the angled end of the ipsilateral sheath, by pushing the first wire through the ipsilateral sheath from an other end of the ipsilateral sheath, such that the first magnet exits the endograft stent through a gate; inserting a short sheath into the aneurysm sac through a second iliac artery, wherein the short sheath houses a second wire, and wherein a second magnet is secured to an end of the second wire and positioned at an opening of the short sheath inside the aneurysm sac; and coupling the first magnet to the second magnet such that the gate is aligned with the second iliac artery.

The present invention also provides an apparatus for installing an endograft stent in an aorta including the endograft stent, positioned inside an aneurysm sac of the aorta, wherein the endograft stent includes a long limb positioned in a first iliac artery of the aorta; an ipsilateral sheath, having an angled end inserted into the long limb through the first iliac artery; a first wire housed inside the ipsilateral sheath; a first magnet secured to an end of the first wire and positioned at an opening of the angled end of the ipsilateral sheath; a push rod, for pushing the first wire through the ipsilateral sheath from an other end of the ipsilateral sheath, such that the first magnet exits the endograft stent through a gate; a short sheath, having an end inserted into the aneurysm sac through a second iliac artery; a second wire housed inside the short sheath; and a second magnet secured to an end of the second wire and positioned at an opening of the short sheath inside the aneurysm sac, wherein the second magnet couples with the exited first magnet such that the gate is aligned with the second iliac artery.

A method for installing an endograft stent in an aorta according to a second embodiment of the present invention is also provided, including placing the endograft stent in an aneurysm sac of the aorta with a long limb of the endograft stent in a first iliac artery of the aorta; inserting an angled end of a first wire into the long limb, through the first iliac artery, wherein a first magnet is secured to an end of the first wire; inserting a second wire into aneurysm sac through a second iliac artery, wherein a second magnet is secured to an end of the second wire and positioned inside the aneurysm sac; and coupling the first magnet to the second magnet such that the gate is aligned with the second iliac artery.

An apparatus is also provided for installing an endograft stent in an aorta according to the second embodiment of the present invention including the endograft stent, positioned inside an aneurysm sac of the aorta, wherein the endograft stent includes a long limb positioned in a first iliac artery of the aorta; a first wire, having an angled end inserted into the long limb through the first iliac artery; a first magnet secured to an end of the first wire and positioned at the angled end of the first wire; a push rod, for pushing the first wire from an other end of the first wire, such that the first magnet exits the endograft stent through a gate; a second wire, having an end inserted into the aneurysm sac through a second iliac artery; and a second magnet secured to an end of the second wire and positioned inside the aneurysm sac, wherein the second magnet couples with the exited first magnet such that the gate is aligned with the second iliac artery.

### BRIEF DESCRIPTION OF THE DRAWINGS

The above and other objects, features and advantages of certain exemplary embodiments of the present invention will be more apparent from the following detailed description taken in conjunction with the accompanying drawings, in which:

FIG. 1 is a view of a conventional endograft stent positioned inside an aorta;

FIG. 2 is a view of an apparatus for installing an endograft stent in an aorta according to a first exemplary embodiment of the present invention; and

FIG. 3 is a schematic view of an apparatus according to a second exemplary embodiment of the present invention.

### DETAILED DESCRIPTION OF THE PREFERRED EMBODIMENTS

The following detailed description of preferred embodiments of the invention will be made in reference to the accompanying drawings. In describing the invention, explanation about related functions or constructions known in the art are omitted for the sake of clearness in understanding the concept of the invention, to avoid obscuring the invention with unnecessary detail.

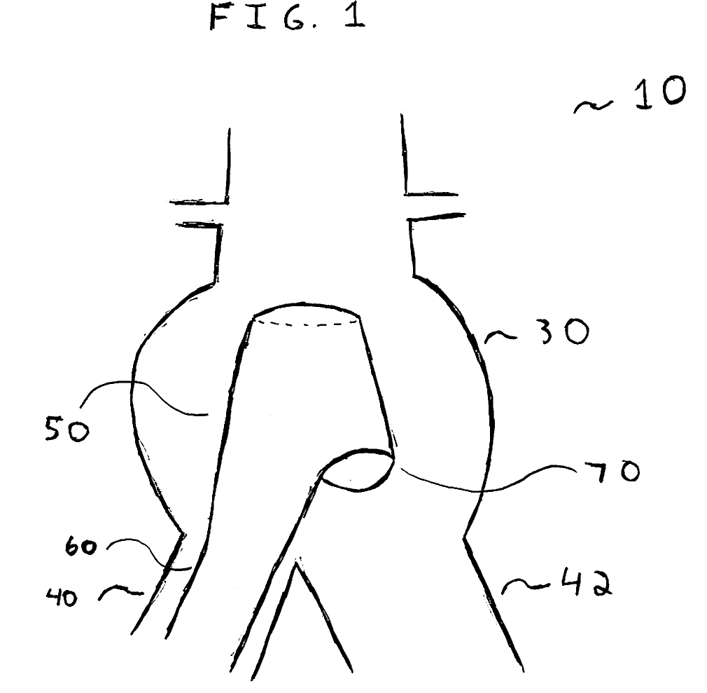

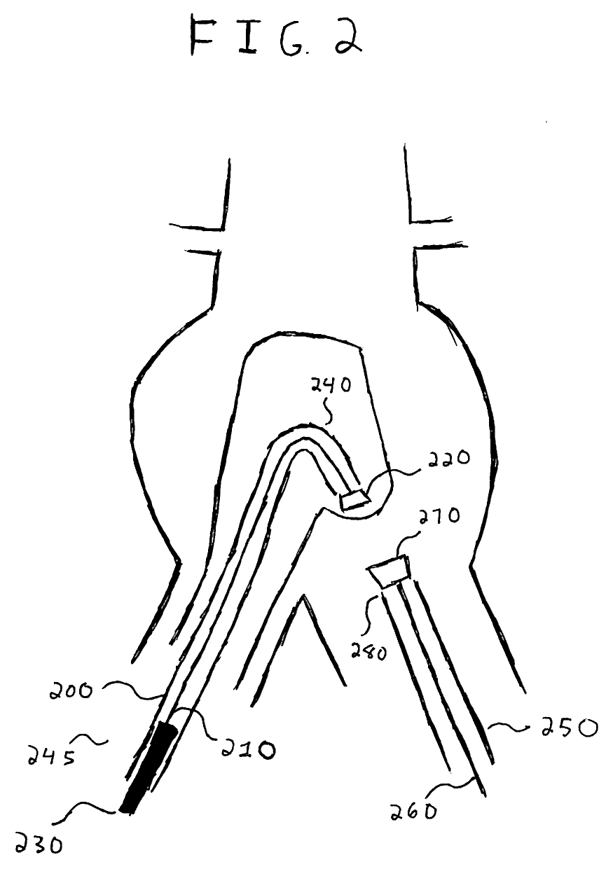

As shown in FIG. 2, with reference to FIG. 1, in a preferred embodiment of the present invention, an apparatus for installing an endograft stent 50 in an aorta 10 includes an ipsilateral sheath 200, a first wire 210, a first rod magnet 220, a push rod 230, a short sheath 250, a second wire 260, and a second rod magnet 270.

Once an endograft stent 50 is positioned in an aneurysm sac 30 of the aorta 10 with a long limb 60 of the endograft stent 50 in a first iliac artery 40 of the aorta 10, an angled end 240 of the ipsilateral sheath 200 is inserted into the long limb 60, through the first iliac artery 40. The ipsilateral sheath 200 houses the first wire 210, and the first magnet 220 is secured by glue or the like to an end of the first wire 210, such that the first magnet 220 is positioned at an opening of the angled end 240 of the ipsilateral sheath 20.

After the ipsilateral sheath 200 is inserted into the long limb 60, the first magnet 220 is pushed out of the opening of the angled end 240 of the ipsilateral sheath 200, by pushing the first wire 210 through the ipsilateral sheath 200 from an other end 245 of the ipsilateral sheath 200. As the first wire 210 is pushed through the ipsilateral sheath 200, the first magnet 210 exits the endograft stent 50 through the gate 70.

A short sheath 250 is inserted into aneurysm sac 30 through the second iliac artery 42. The short sheath 250 houses a second wire 260 and a second rod magnet 270 is secured to an end of the second wire 260, such that the second magnet 270 is positioned at an opening 280 of the short sheath 250 inside the aneurysm sac 30.

As both the first and second rod magnets 220 and 270 are positioned inside the sac 30, the magnets 220 and 270 couple with each other, while aligning the gate 70 with the second iliac artery 42.

The ipsilateral sheath 200 may be a 16FR sheath, and the angle at the angled end 240 of the ipsilateral sheath 200 may be set to approximately 30 degrees, allowing the ipsilateral sheath 200 to sit on a bifurcation of the endograft stent 50. However, the present invention is not limited to this size and angle. The first wire 210 may be composed of a soft flexible material, allowing the first wire 210 to pass through the angle of the ipsilateral sheath 200. The second wire 270 of the short sheath 250 may be composed of a rigid inflexible material, to facilitate the alignment of the gate 70 with the second iliac artery 42. However, the present invention is not limited to a rigid material.

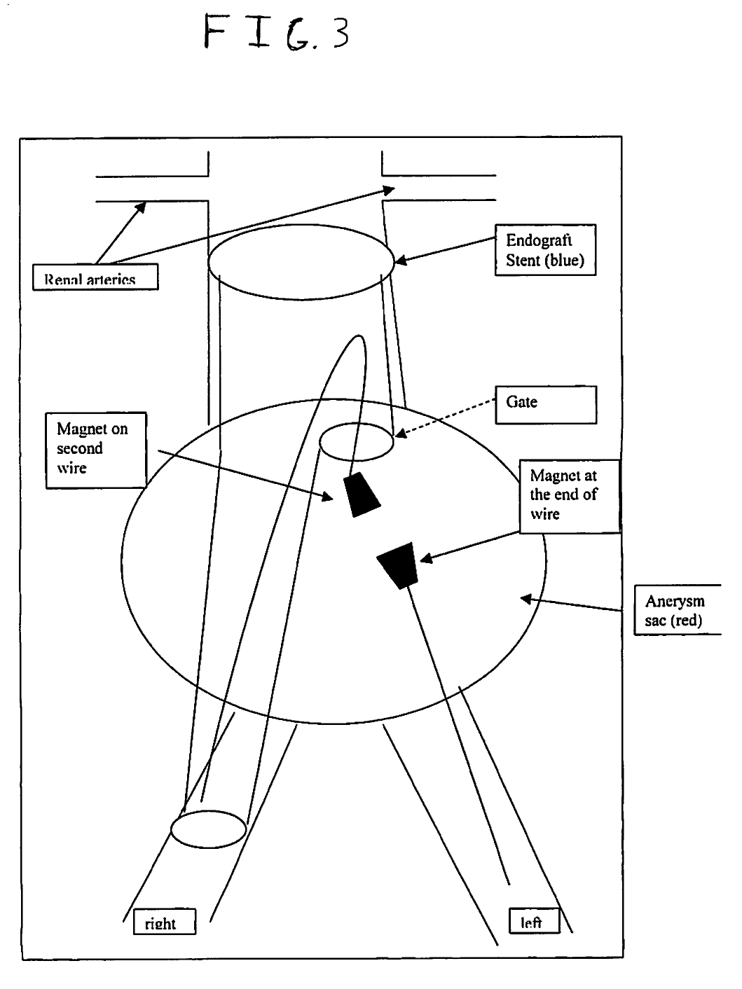

FIG. 3 is a schematic view of an apparatus according to a second exemplary embodiment of the present invention.

The apparatus in FIG. 3 is works in a manner similar to the apparatus shown in FIGS. 1 and 2, except that the first and second wires 210 and 260 are inserted without the ipsilateral sheath 200 and the short sheath 250.

According to a third exemplary embodiment of the invention of the present invention (not shown), a method and apparatus for installing an endograft stent operates in a manner similar to the first embodiment of the present invention, except, in place of the first and second rod magnets 220 and 270, as shown in FIG. 2, both the first and second wires 220, 260 are composed of a magnetic material, and at least one of the first and second wires 220, 260 is magnetized, allowing the first and second wires to couple without using separate magnet pieces.

According to a fourth exemplary embodiment of the invention of the present invention (not shown), a method and apparatus for installing an endograft stent operates in a manner similar to the second embodiment of the present invention, except, in place of the first and second rod magnets 220 and 270, as shown in FIG. 2, both the first and second wires 220, 260 are composed of a magnetic material, and at least one of the first and second wires 220, 260 is magnetized, allowing the first and second wires to couple without using separate magnet pieces.

While this invention has been particularly shown and described with reference to preferred embodiments thereof, it will be understood by those skilled in the art that various changes in form and details may be made therein without departing from the scope of the invention encompassed by the appended claims.

What is claimed is:

1. A method for installing an endograft stent in an aorta comprising:
placing the endograft stent in an aneurysm sac of the aorta with a long limb of the endograft stent in a first iliac artery of the aorta;
inserting an angled end of an ipsilateral sheath into the long limb, through the first iliac artery, wherein the ipsilateral sheath houses a first wire, and wherein a first magnet is secured to an end of the first wire and positioned at an opening of the angled end of the ipsilateral sheath;
pushing the first magnet out of the opening of the angled end of the ipsilateral sheath, by pushing the first wire through the ipsilateral sheath from an other end of the ipsilateral sheath, such that the first magnet exits the endograft stent through a gate;
inserting a short sheath into the aneurysm sac through a second iliac artery, wherein the short sheath houses a second wire, and wherein a second magnet is secured to an end of the second wire and positioned at an opening of the short sheath inside the aneurysm sac; and
coupling the first magnet to the second magnet such that the gate is aligned with the second iliac artery.

2. The method of claim 1, wherein the angled end of the ipsilateral sheath is bent to an angle of approximately 30 degrees.

3. The method of claim 1, wherein the first wire has a flexible structure.

4. The method of claim 1, wherein the second wire has a rigid structure.

5. An apparatus for installing an endograft stent in an aorta comprising:
the endograft stent, positioned inside an aneurysm sac of the aorta, wherein the endograft stent includes a long limb positioned in a first iliac artery of the aorta;
an ipsilateral sheath, having an angled end inserted into the long limb through the first iliac artery;
a first wire housed inside the ipsilateral sheath;
a first magnet secured to an end of the first wire and positioned at an opening of the angled end of the ipsilateral sheath;
a push rod, for pushing the first wire through the ipsilateral sheath from an other end of the ipsilateral sheath, such that the first magnet exits the endograft stent through a gate;
a short sheath, having an end inserted into the aneurysm sac through a second iliac artery;
a second wire housed inside the short sheath; and
a second magnet secured to an end of the second wire and positioned at an opening of the short sheath inside the aneurysm sac, wherein the second magnet couples with the exited first magnet to align the gate with the second iliac artery.

6. The apparatus of claim 5, wherein the angled end of the ipsilateral sheath is bent to an angle of approximately 30 degrees.

7. The apparatus of claim 5, wherein the first wire has a flexible structure.

8. The apparatus of claim 5, wherein the second wire has a rigid structure.

9. A method for installing an endograft stent in an aorta comprising:
placing the endograft stent in an aneurysm sac of the aorta with a long limb of the endograft stent in a first iliac artery of the aorta;
inserting an angled end of a first wire into the long limb, through the first iliac artery, wherein a first magnet is secured to an end of the first wire;
inserting a second wire into aneurysm sac through a second iliac artery, wherein a second magnet is secured to an end of the second wire and positioned inside the aneurysm sac; and
coupling the first magnet to the second magnet such that the gate is aligned with the second iliac artery.

10. The method of claim 9, wherein the angled end of the first wire is bent to an angle of approximately 30 degrees.

11. The method of claim 9, wherein the first wire has a flexible structure.

12. The method of claim 9, wherein the second wire has a rigid structure.

13. An apparatus for installing an endograft stent in an aorta comprising:
the endograft stent, positioned inside an aneurysm sac of the aorta, wherein the endograft stent includes a long limb positioned in a first iliac artery of the aorta;
a first wire, having an angled end inserted into the long limb through the first iliac artery;
a first magnet secured to an end of the first wire and positioned at the angled end of the first wire;
a push rod, for pushing the first wire from an other end of the first wire, such that the first magnet exits the endograft stent through a gate;
a second wire, having an end inserted into the aneurysm sac through a second iliac artery; and
a second magnet secured to an end of the second wire and positioned inside the aneurysm sac, wherein the second magnet couples with the exited first magnet to align the gate with the second iliac artery.

14. The apparatus of claim 13, wherein the angled end of the first wire is bent to an angle of approximately 30 degrees.

15. The apparatus of claim 13, wherein the first wire has a flexible structure.

16. The apparatus of claim 13, wherein the second wire has a rigid structure.
